(19) United States
(12) Patent Application Publication
(10) Pub. No.: US 2010/0278798 A1
(43) Pub. Date: Nov. 4, 2010

(54) METHODS AND SYSTEMS FOR FORMING BIOCOMPATIBLE MATERIALS

(75) Inventors: Samuel K. Sia, New York, NY (US); Brian Michael Gillette, Bronx, NY (US)

Correspondence Address: MILES & STOCKBRIDGE PC, 1751 PINNACLE DRIVE, SUITE 500, MCLEAN, VA, 22102-3833, US

(73) Assignee: The Trustees of Columbia University in the City of New York, New York, NY (US)

(21) Appl. No.: 12/675,181

(22) PCT Filed: Sep. 29, 2008
(86) PCT No.: PCT/US08/78197

§ 371 Date: Jul. 8, 2010

Related U.S. Application Data
(63) Continuation of application No. 61/056,309, filed on May 27, 2008.
(60) Provisional application No. 60/975,805, filed on Sep. 27, 2007.

(51) Int. Cl. A61K 38/43; C12N 5/07; A61K 38/39; A61P 17/02

(52) U.S. Cl. 424/94.1; 435/398; 514/17.2

(57) ABSTRACT

Methods and systems forming biocompatible materials are disclosed herein. Forming a biocompatible material may include contacting a liquid, having a linking material, with an adjoining material having embedded therein a nucleating material that causes the linking material to nucleate and grow into the liquid. After a time sufficient to cause the linking material to grow substantially from the nucleating material into a space occupied by the liquid, the liquid may be solidified to form a solid such that the linking material secures the solid to the adjoining material.

### CROSS REFERENCE TO RELATED APPLICATIONS

This application claims priority to U.S. Provisional Patent Application No. 60/975,805, filed on Sep. 27, 2007, and U.S. Provisional Patent Application No. 61/056,309, filed on May 27, 2008, the entirety of each of the disclosures of which are explicitly incorporated by reference herein.

### BACKGROUND

Fabricated three-dimensional (3D) extracellular matrices (ECMs) can be used to mimic the often inhomogeneous and anisotropic properties of native tissues and to construct in vitro cellular environments. Since these 3D ECMs provide physiologically relevant cellular environments, they can be used to study tissue morphogenesis as well as to engineer tissue. For example, 3D collagen, Matrigel® (BD Biosciences: gelatinous protein mixture secreted by mouse tumor cells), and fibrin matrices can be used for analyzing the mechanisms of epithelial branching morphogenesis and endothelial cell capillary morphogenesis as well as for engineering vascular and cardiac tissues. To this end, bulk isotropic 3D matrices have been employed in which cells are randomly dispersed. However, these bulk structures offer limited control over spatial parameters that may influence cell assembly and tissue architecture. Precise control over cell positioning and ECM composition (on the order of tens of microns or less) enables improved regulation of the driving forces behind tissue morphogenesis, such as paracrine signaling, interactions between cells and ECMs, and cell to cell contact. For example, control of epithelial organoid geometry in microscale patterned 3D matrices allows regulation of epithelial branching patterns by autocrine factor gradients. Thus, a wide variety of methods, such as micromolding and dielectrophoresis, have been used to control microscale geometry, fluid flow and cell localization within 3D matrices. However, cellular contraction, or other modes of strain, of fibrous natural ECMs, such as fibrin and collagen I, can detach these matrices from their surroundings and adversely affect the geometry, including destruction thereof.

### SUMMARY

Methods for and systems forming biocompatible materials are disclosed herein.

In some methods, forming a biocompatible material includes (i.e., comprises) contacting a liquid, having (i.e., comprising) a linking material, with an adjoining material having embedded therein a nucleating material that causes the linking material to nucleate and grow into the liquid, and, after a time sufficient to cause the linking material to grow substantially from the nucleating material into a space occupied by the liquid, solidifying the liquid to form a solid such that the linking material secures the solid to the adjoining material.

In an aspect of some methods, the solid is a gel and the solidifying includes gelling the liquid.

In an aspect of some methods, the liquid and the adjoining material include at least one culture medium and live cells.

In an aspect of some methods, the liquid and the solid formed therefrom include at least one of collagen, fibrin, alginate, and Matrigel®.

In an aspect of some methods, the linking material includes at least one of engineered and natural fiber macromolecules.

In an aspect of some methods, the linking material includes at least one of collagen, fibrin, elastin, laminin, and fibronectin.

In an aspect of some methods, the nucleating material is the same essential molecular structure as the linking material.

In an aspect of some methods, the linking and nucleating materials include collagen.

In an aspect of some methods, the adjoining material includes living tissue.

In an aspect of some methods the adjoining material and the solid are essentially the same composition having different cell types.

In an aspect of some methods, forming a biocompatible material further includes contacting a further liquid having a linking material with the solid such that the linking material in the solid nucleates the linking material in the further liquid, and solidifying the further liquid to form a further solid.

In an aspect of some methods, the space occupied by the liquid includes a flow channel and the contacting includes flowing the liquid through the flow channel. In an aspect of some methods, the flow channel is part of a channel network and has a width of less than 100 microns.

In some methods, forming a biocompatible material includes stemming fibers in at least one liquid containing a soluble fibrilizing material from fibers in an adjacent material at least one surface adjacent to the at least one liquid such that the fibers are continuous across the at least one surface from the adjacent material to the at least one liquid and gelling the at least one liquid to hold fast, stemmed fibers in gelled liquid resulting from the gelling.

In an aspect of some methods, the at least one liquid includes living cells.

In an aspect of some methods, the gelling includes crosslinking a material in the liquid that is a different material from the fibrilizing material.

In an aspect of some methods, the fibrilizing material includes collagen and the gelling includes gelling an agent other than collagen.

In some methods, forming a biocompatible material includes supporting cells in a solid containing a solidifying material and a fiber network, modifying a property of the solidifying material to permit the cells to move and/or grow, and thereafter restoring the property of the solidifying material to inhibit cells from moving and/or growing while leaving the fiber network substantially intact.

In an aspect of some methods, the supporting includes contacting a liquid, having the cells and the second fibers, with an adjoining material having embedded therein the second fibers thereby causing the fibers to nucleate at the adjoining material second fibers and grow into the liquid and after a time sufficient to cause the second fibers to grow substantially into a space occupied by the liquid, mutually linking the first fibers to gel the liquid and secure the gel to the adjoining material.

In an aspect of some methods, the fiber network includes collagen and or fibrin.

In an aspect of some methods, at least some of the gelled layers are of different materials including at least one incorporating different types of living cells.

In an aspect of some methods, at least some of the gelled layers have living cells therein, the living cells in one of the at least some gelled layers being different from the living cells in another of the at least some gelled layers.

In some methods, forming a biocompatible material includes providing at least one gel body containing first cells and fibers forming a scaffold within the gel, the fibers joining the gel to an adjoining material, and reversibly altering a property of the gel material forming the at least one gel body to permit the first cells to grow and/or move while the first cells continue to be supported by the fibers forming the scaffold.

In an aspect of some methods, the forming a biocompatible material further includes restoring the property of the gel material forming the at least one gel body after an interval required for the growth and/or movement of the first cells.

In an aspect of some methods, the property includes a crosslinking of material forming the gel material of the at least one gel body.

In an aspect of some methods, the gel includes one of alginate and agarose.

In an aspect of some methods, the gel includes alginate and the reversibly altering includes applying calcium chloride to the at least one gel body and the restoring includes applying a calcium chelator thereto.

In an aspect of some methods, the at least one gel body is one of multiple layers of gel.

In an aspect of some methods, the adjoining material is a gel a different material from the at least one gel body.

In an aspect of some methods, the adjoining material is a gel containing second cells of a different type from the first cells.

In an aspect of some methods, the property includes a mechanical property.

In some materials, a biocompatible material includes a first gel material having at least one channel therein, and a second gel material in the at least one channel, wherein the second gel material is joined to the first gel material at an interface therebetween by fibers.

In an aspect of some materials, at least the second gel material contains living cells.

In an aspect of some materials, the fibers extend through the interface into the first and second gel materials to join the first and second gel materials together.

In an aspect of some materials, a substantial fraction of the fibers extending through the interface in the first and second gel materials are directed at angles of approximately 90 degrees relative to the interface.

In an aspect of some materials, at least a portion of the at least one channel has a width less than 100 microns.

In an aspect of some materials, the at least one channel includes a network of channels.

In an aspect of some materials, the first and second gel materials are of different materials.

In an aspect of some materials, the first and second gel materials have different respective live cells.

In some methods, linking a matrix phase to a second material phase includes flowing a liquid gel precursor containing a fibrilizing material adjacent an adjoining material, the adjoining material having, at an interface between the liquid and adjoining material, a nucleating material which is adapted to nucleate growth of the fibrilizing material, assembling fibers in the gel precursor at the interface to form a fiber network in the liquid gel precursor, and thereafter gelling the gel precursor.

In an aspect of some methods, the gel has living cells therein.

In an aspect of some methods, the adjoining material includes living tissue.

In an aspect of some methods, the gel precursor includes a hormone effective to stimulate a change in growth of living cells in the adjoining material.

In an aspect of some methods, the adjoining material includes living tissue, the linking a matrix phase to a second material phase further including enzymatically treating the living tissue to enhance the exposure of the nucleating material to the fibrilizing material.

In an aspect of some methods, the adjoining material includes living tissue, the linking a matrix phase to a second material phase further including, prior to the flowing, enzymatically treating the living tissue to enhance the exposure of the nucleating material to the fibrilizing material.

In an aspect of some methods, the adjoining material is a gel including a network of microchannels and the flowing includes flowing the liquid gel precursor through at least a portion of the network.

In an aspect of some methods, the nucleating material includes existing fibers, of the same material as the fibrilizing material, extending into the adjoining material, the assembling including extending the existing fibers to form the fiber network.

In some systems, a medical treatment kit includes materials that may be combined to form, or, a liquid composition containing a gel precursor and soluble collagen, and a biocompatible gelling agent effective to solidify the gel precursor.

In an aspect of some systems, a medical treatment kit further includes an enzyme effective to enhance the exposure of collagen in living tissue to act as nucleation sites for the soluble collagen.

In some materials, a biocompatible material includes a first material of a first composition, a second material of a second composition adjacent to and in contact with the first material, and fibers extending between the first and second materials across a first interface therebetween, wherein the first material is principally a hydrogel.

In an aspect of some materials, a biocompatible material further includes a third material in contact with the first on a side opposite the first interface in which the fibers continue across a second interface between the first and third materials.

In an aspect of some materials, the second material includes living tissue.

In an aspect of some materials, the first material includes living cells, the fibers having a number, type, and orientation effective to prevent detachment of the first interface in response to a growth of the living cells.

In an aspect of some materials, a biocompatible material further includes a third material adjacent to and in contact with the second material at a second interface therewith, and fibers extending between the second and third materials across the second interface.

In an aspect of some materials, a substantial fraction of the fibers extending across the first interface have angles of approximately 90 degrees with respect to the first interface.

In some methods, forming a biocompatible material includes stemming fibers in at least one liquid containing a soluble fibrilizing material from fibers in an adjacent material at least one surface adjacent to the at least one liquid such that the fibers are continuous across the at least one surface from the adjacent material to the at least one liquid, and solidifying the at least one liquid to hold fast, stemmed fibers in solidified liquid resulting from the solidifying.

### BRIEF DESCRIPTION OF THE DRAWINGS

FIG. 1 is a schematic diagram showing a microfabrication process for constructs for patterning integrated matrix phases.

FIG. 2 is a schematic diagram of a pattern for a construct of integrated matrix phases.

FIGS. 3A-3C are schematic diagrams showing magnified views of the interface between matrix phases during the formation of a construct of integrated matrix phases.

FIG. 4 is a time-lapse image comparison of fiber assembly at the interface between matrix phases.

FIG. 5A is an image of fiber assembly at the interface between a collagen matrix phase and a collagen-doped alginate matrix phase.

FIG. 5B is an image of fiber assembly at the interface between a collagen matrix phase and a pure alginate matrix phase.

FIG. 6A is an image of fiber assembly at the interface between a collagen-doped Matrigel® matrix phase and a collagen-doped alginate matrix phase.

FIG. 6B is an image of fiber assembly at the interface between a pure Matrigel® matrix phase and a collagen-doped alginate matrix phase.

FIG. 7A is an image of fiber assembly at the interface between a collagen-doped fibrin matrix phase and a collagen-doped alginate matrix phase.

FIG. 7B is an image of fiber assembly at the interface between a pure fibrin matrix phase and a collagen-doped alginate matrix phase.

FIG. 8A is an image of a pattern for a construct of integrated matrix phases with living cells isolated in the different phases.

FIG. 8B is an image of a pattern for a construct of integrated matrix phases with living cells isolated in the different phases.

FIG. 8C is a confocal reflectance microscopy reconstruction of a portion of a patterned cell-seeded matrix phase material.

FIG. 8D is a confocal reflectance microscopy reconstruction of a portion of a linking-material-doped bulk matrix phase after fabrication.

FIG. 9A is an image showing the morphology of living cells inside a bulk matrix phase at 15 minutes after exposure of the bulk matrix phase to a ungelling agent.

FIG. 9B is an image showing the morphology of living cells inside a bulk matrix phase at 3 hours after exposure of the bulk matrix phase to a ungelling agent.

FIGS. 10A-10B are images showing the morphology of living cells inside a bulk matrix phase at 15 minutes and 3 hours without exposure of the bulk matrix phase to a ungelling agent.

FIG. 11 shows graphs quantifying the cell aspect ratio and cell area for the cells in the bulk matrix phases of FIGS. 9-10.

FIG. 12 is an image showing a living cell structure in an integrated matrix phase after 22 days of culture.

FIG. 13A is a diagram comparing cell behavior in a single-component matrix phase.

FIG. 13B is a diagram comparing cell behavior in a composite matrix phase before and after reversibly un-crosslinking.

FIG. 14 is a schematic diagram showing a process flow for reversibly crosslinking and un-crosslinking a composite matrix phase.

FIG. 15 shows images of cell behavior at 0, 3, 6, and 9 hours in composite matrix phases that had been crosslinked, un-crosslinked, and selectively crosslinked and un-crosslinked.

FIG. 16A is an image of a pattern for a construct of integrated matrix phases with living cells isolated in the different phases.

FIG. 16B is a magnified image of a portion of FIG. 16A showing cells isolated in the different phases, according to some embodiments of the disclosed subject matter.

FIG. 17 is a schematic diagram of a construct of integrated matrix phases with multiple layers.

FIG. 18 is a schematic diagram showing a fabrication process for orienting fibers in the constructs in a particular orientation

FIG. 19 is a schematic diagram showing a process for spatial control of matrix phase porosity using optical techniques.

### DETAILED DESCRIPTION

Fabricated micro-scale three-dimensional (3D) extracellular matrices (ECMs) can be used to mimic properties of native tissues and to construct in vitro cellular environments, while providing precise control over cell positioning and ECM composition (on the order of tens of microns or less). However, cellular contraction can detach the matrix phases from their surroundings and thereby adversely affect or destroy the geometry.

A preferred method includes in situ fiber assembly to anchor together multiple phases of matrix material to resist cellular contractile forces and other strain. The fibers in one of the matrix material phases can be grown from preformed fibers, which can serve as a nucleation template for the new fiber growth, in another matrix material phase at an interface between the two material phases. The fibers serve to securely attach the matrix material phases to each other. Thus, the fiber-mediated interfacing allows the microfabricated 3D matrices to maintain stable interfaces such that the individual material phases do not detach from each other over a long-term culture despite the induced strain by the cells contained in the matrix phase.

The fiber-mediated interfacing enables construction of well-defined and stable patterns of a variety of matrix material phases formed by diverse mechanisms, such as temperature-mediated, ion-mediated, and enzyme-mediated crosslinking. First and second matrix material phases can be formed by different crosslinking methods and then joined together via in situ fiber assembly. Each material phase can be seeded with cells and designed to permit cell spreading. Each material phase can also be specifically adapted to different functions. Thus, different types of matrix material phases may be anchored together against cellular contraction. This enables high-resolution control of cell and matrix phase material localization to mimic the inhomogeneous and anisotropic properties of natural tissues.

The integration of various matrix material phases with different porosity, cell adhesion, and cross-linking characteristics can allow independent control of cell dynamics. In particular, one of the integrated matrix material phases can be reversibly cross-linked such that porosity of the matrix material may be adjusted between a large pore state that allows cell migration and a small pore state that restricts cell migration. Thus, the method can selectively permit or restrict mobility of encapsulated cells. Furthermore, the integrated matrix material phases provided by the method can act as diffusively permeable migration barrier to cells in interfaced matrix material phases to provide spatial cues that can influence cell behavior.

The fiber-mediated interfacing can be adapted to in vivo applications, such in vivo tissue growth, adhesion of matrix materials to tissues, and wound care.

While the method discussed herein is addressed to certain types of ECM materials, the method is applicable to a wide range of matrix phase material, as would be readily appreciated by one of ordinary skill in the applicable arts. For example, the method can be applied to any type of hydrogel, such as naturally occurring ECM materials. Such hydrogels may include, but are not limited to, collagen, fibrin, alginate, and Matrigel® (a gelatinous protein mixture secreted by mouse tumor cells and marketed by BD Biosciences).

Furthermore, although the method is discussed herein using collagen I precursors to form collagen I fibers, the method is applicable to a wide variety of linking materials, that one of ordinary skill in the applicable arts would readily appreciate. For example, the method can be employed with any type of fibrous macromolecule capable of nucleating, such as fibrous proteins. Such fibrous proteins may include, but are not limited to, collagen, fibrin, elastin, laminin, and fibronectin.

In a generalized process for securing a gel to an adjoining material, an adjoining material can provided with nucleation sites at a boundary surface. The adjoining material can be a matrix material phase, such as a gel, a hydrogel, or an ECM. Alternatively, the adjoining material can be living tissue, such as living tissue in vivo. For example, the adjoining material can be skin. A gel precursor with linking material precursors is flowed past the boundary surface of the adjoining material. Although referred to as separate components, the linking material precursors and the gel precursor may be the same material. For example, the gel and linking material precursors may be collagen I.

As the gel precursor flows past the boundary surface, the linking material precursors begin to assemble at the nucleation sites to form a linking or joining material. The linking material can be in the form of strands or fibers. The linking material extends into the gel precursor away from the boundary surface. A property of the gel precursor can be changed to thereby gel the gel precursor to form the gel. The linking material extends into the gel away from the boundary surface and in effect secures the gel to the adjoining material. Although described separately, the formation of the linking material and the formation of the gel from their respective precursors may occur simultaneously.

The nucleation sites provided at the boundary surface of the adjoining material can be formed by preformed linking material in the adjoining material. The preformed linking material can be made from the same linking material precursor as the linking material precursor in the gel. Thus, linking material can be formed extending across the boundary surface and extending substantially into both the adjoining material and the gel.

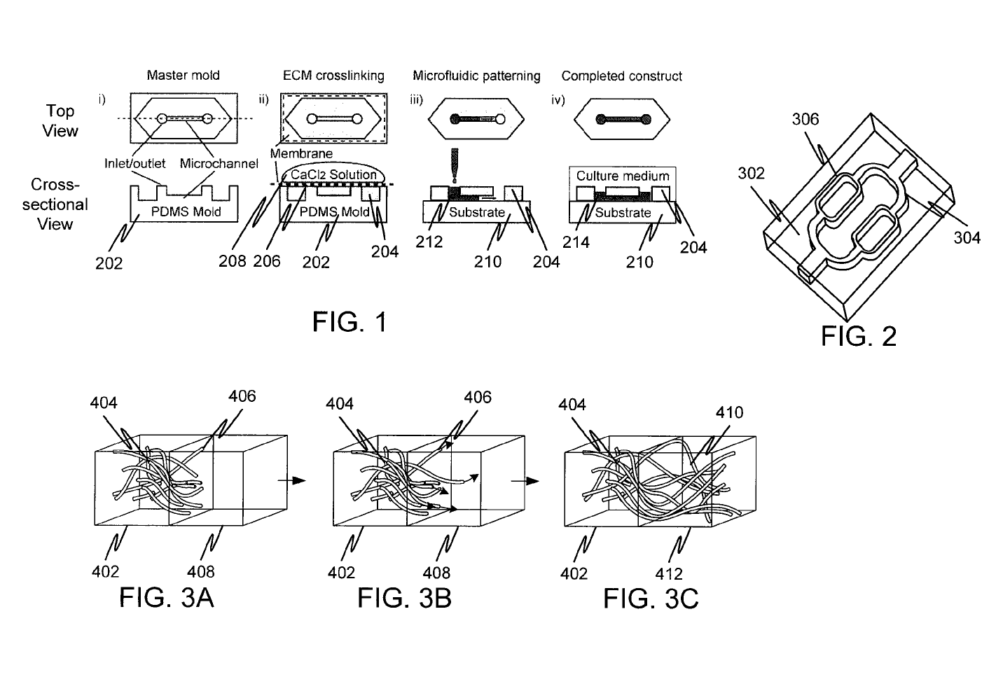

FIG. 1 is a schematic diagram showing a process for constructing integrated matrix phases. The construct can have a microfluidically patterned phase and a bulk matrix phase, such as that shown in FIG. 2. Various channels can be formed in the bulk matrix phase, which then define the geometry of the patterned matrix phase. For example, with reference to FIG. 2, the bulk matrix phase material 302 can be cast to have a network of microfluidic channels 304 that can be filled with patterned matrix phase material 306.

With reference to FIG. 1(i), a mold 202 is created. The mold can be made by any manner known in the art. For example, master molds can be developed using multilayer photolithography, silicon micromachining, laser machining, or rapid prototyping techniques. Referring to FIG. 1(ii), alginate-based solutions are flowed (e.g., 30-100 μl depending on mold design) into the mold 202 and covered with a semi-permeable dialysis membrane 206. Alginate with high guluronic acid content and medium viscosity produces physically robust constructs at low alginate concentrations (1-3% w/v) appropriate for 3D culture of mammalian cells.

The alginate phase can be doped with collagen and allowed to form a collagen fiber network therein by increasing temperature. As shown in FIG. 3A, the collagen I forms a fiber network 404 in the bulk matrix phase 402, which will later serve as a nucleation template 406 for assembly of fibers in the adjacent patterned phase 408. To prepare the collagen-doped alginate mixtures, a 10 mg/ml collagen in 3 mM HCL solution can be neutralized using 2% v/v 1N NaOH, then mixed on ice 3:1 with a 3-8% alginate solution that was mixed with cell suspension or medium to yield final concentrations of 3-6 mg/ml collagen and 6-30 mg/ml alginate. The resulting collagen-doped alginate solutions in the mold can be incubated at room temperature for 40 minutes to crosslink collagen.

The alginate may then be gelled (by ionic crosslinking) around the collagen fiber network to complete formation of the bulk matrix phase. The alginate solution in the mold 202 is cross-linked by flowing a 60 mM CaCl₂ solution 208 in serum-free medium or 15 mM HEPES/150 mM NaCl buffer onto the membrane 206 for 20-30 minutes. Calcium ions from solution 208 diffuse across the membrane and bind with multiple alginate molecules to create a nanoporous matrix. This matrix serves as the bulk matrix phase material 204 for patterning the second matrix phase material.

The bulk matrix phase is preferably formed by first gelling collagen (e.g., by increasing temperature) and then alginate (e.g., by ionic crosslinking). After removing the collagen-alginate construct 204 from the mold 202, the matrix phase 204 may be placed in a media bath to equilibrate calcium ion concentration and further incubated in a humidified, 37° C., 5% v/v CO₂ incubator prior to fabricating the patterned matrix phase. Referring to FIG. 1(iii), the alginate matrix phase 204 may be plated in an 8-well untreated polystyrene plate or on a coverglass as a substrate 210. The substrate 210 seals the bottom of the channel during microfluidic formation of the patterned matrix phase. A cell-seeded ECM precursor 212, for example, collagen I, is flowed into the inlet of the channels. The flow may proceed through the channels by hydrostatic pressure.

With reference to FIG. 3B, as the precursor 408 flows adjacent to an interface with the bulk matrix phase 402, the fibers 404 formed in the bulk matrix phase 402 serve as nucleation sites 406 for the formation of additional fibers in the patterned matrix phase. As the temperature is increased, collagen precursors in the patterned matrix phase nucleate and assemble from exposed collagen fibers at the interface thereby integrating the two matrices. Thus, in gelling of a microfluidically patterned matrix phase, collagen fibers nucleate from the collagen-doped alginate bulk phase and assemble in situ. The resulting structure is one where fibers mechanically linked and/or are continuous across the boundary and can ultimately be anchored in both the bulk and patterned phases. In general, the technique may be used to link adjacent materials irrespective of the shape or configuration of the materials. Also, the linking across the boundary may be a branching connection. The desired effect is associated with mechanical continuity across the boundary and not with any particular quality of the molecular structure.

Referring to FIG. 1(iv), as the fibers form in the patterned matrix phase, the construct may be placed in a humidified, 37° C., 5% CO₂ incubator for 30 minutes to allow collagen polymerization (and simultaneous fiber growth from the nucleation sites) to form the completed patterned matrix phase 214. After formation of the completed construct, 1-3 ml of culture medium can be placed over the construct to supplement the cells therein.

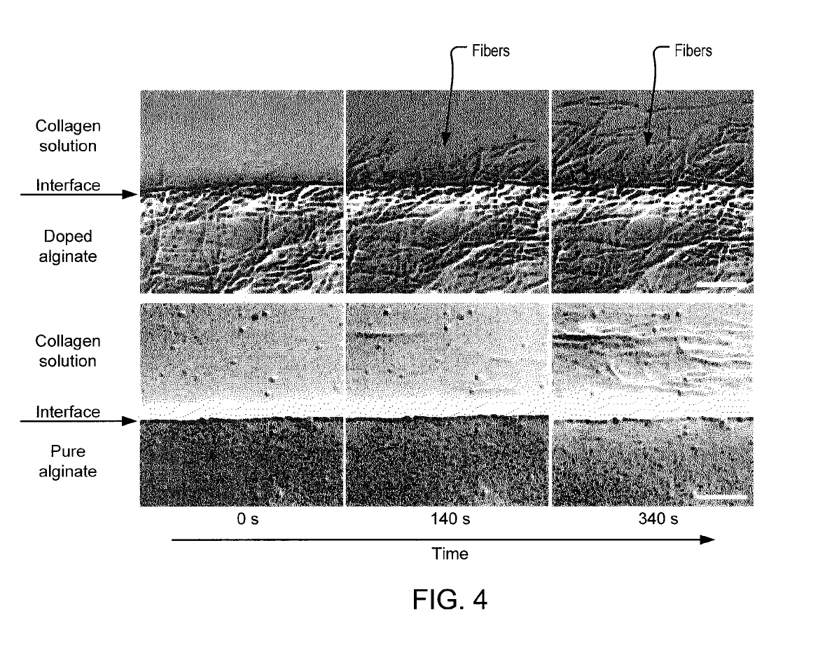

FIG. 4 is a time-lapse image comparison of fiber assembly at the interface between matrix phases. In the top row of images, a patterned collagen phase was formed adjacent to a collagen-doped alginate phase using the disclosed method. In the bottom row of images, a collagen phase was formed adjacent to a pure alginate phase. As is evident from the top row of images, collagen fibers crossed or extended from the bulk phase boundary. However, collagen fibers do not nucleate from a pure alginate bulk phase interface.

This type of fiber integration can occur across a wide range of bulk phase collagen/alginate mass ratios (0.1 to 1) and concentrations of bulk collagen (3-6 mg/ml), alginate (6-30 mg/ml) and patterned collagen (2.5-4 mg/ml). In contrast, when a pure alginate bulk phase was used, the patterned collagen fibers formed in the flowing ECM solution but did not nucleate from the alginate boundary.

On completion of fiber formation, the number of collagen fibers crossing per unit of channel length (those fibers that extended from or crossed the interface) can be in the range of 0.6±0.1 fibers per μm, with the fibers exhibiting an average width of 0.7±0.1 μm for a typical composition of 6 mg/ml of 1:1 w/w collagen-alginate bulk phase and 3 mg/ml of patterned collagen. These values are examples only and are not intended to limit the scope of the claims or the range of applications of which the disclosure is enabling.

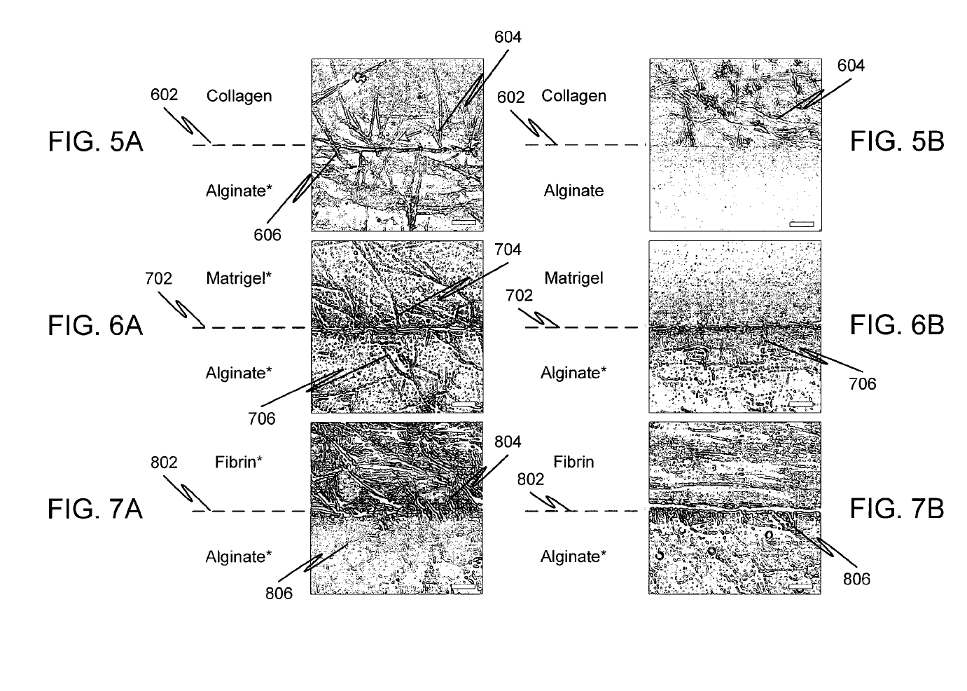

Moreover, the distribution of angles of collagen fibers patterned within a collagen-alginate bulk phase may be biased towards orthogonal angles (orthogonal with respect to a local plane of the interface) compared with angles of fibers within either a bulk collagen-alginate phase or patterned within a pure alginate bulk phase. This angular bias, as well as other fiber morphology, can be influenced by flow dynamics, matrix phase material composition, pH and temperature. Furthermore, in a collagen-alginate bulk phase 606, most (about 80%) of the patterned collagen fibers 604 can be localized to the interface 602 (either extending from or crossing the interface), as shown in FIG. 5A, whereas in a pure alginate bulk phase (in which patterned collagen fibers nucleate from collagen precursors within the channel rather than from the interface), patterned collagen fibers 604 are uniformly distributed throughout the patterned matrix phase, as shown in FIG. 5B. The differential interference contrast image of FIG. 5A of a collagen-alginate bulk phase 606 indicates that many of the collagen fibers 604 in the microchannels cross the channel boundary (arrow) or are anchored to it. However, the differential interference contrast image of FIG. 5B indicates that a pure alginate bulk phase has a sharp boundary with no collagen fibers 604 crossing the phase interface 602.

Materials other than collagen may also be employed for the patterned matrix phase material. Cell-ECM suspensions (containing 10⁶-10⁷ cells per ml) consisting of collagen I (2.5-4 mg/ml) (FIGS. 5A-5B), Matrigel® (4.25 mg/ml) (FIGS. 6A-6B), and fibrinogen (5 mg/ml) (FIGS. 7A-7B) were patterned in the microchannels. Differential interference contrast images indicate that collagen fibers 704 assemble from the bulk collagen-doped alginate phase 706 at the bulk phase interface 702 with patterned collagen-doped Matrigel® (FIG. 6A), but not pure Matrigel® (FIG. 6B). Matrigel® components, primarily collagen IV and laminin, thus do not assemble from the collagen I fibers in the alginate bulk phase. Similarly, differential interference contrast images indicate that collagen fibers 804 assemble from the bulk collagen-doped alginate phase 806 at the bulk phase interface 802 with patterned collagen-doped fibrin (FIG. 7A), but not pure fibrin (FIG. 7B).

These results demonstrate the use of in situ fiber assembly to pattern and interface multiple 3D naturally derived ECMs, including collagen I, Matrigel® and fibrin. The capability for broad flexibility in matrix composition is significant because the influence of composition and stiffness of the local 3D matrix is important for a number of biological processes, such as the development of blood and lymphatic vessels and differentiation of stem cells.

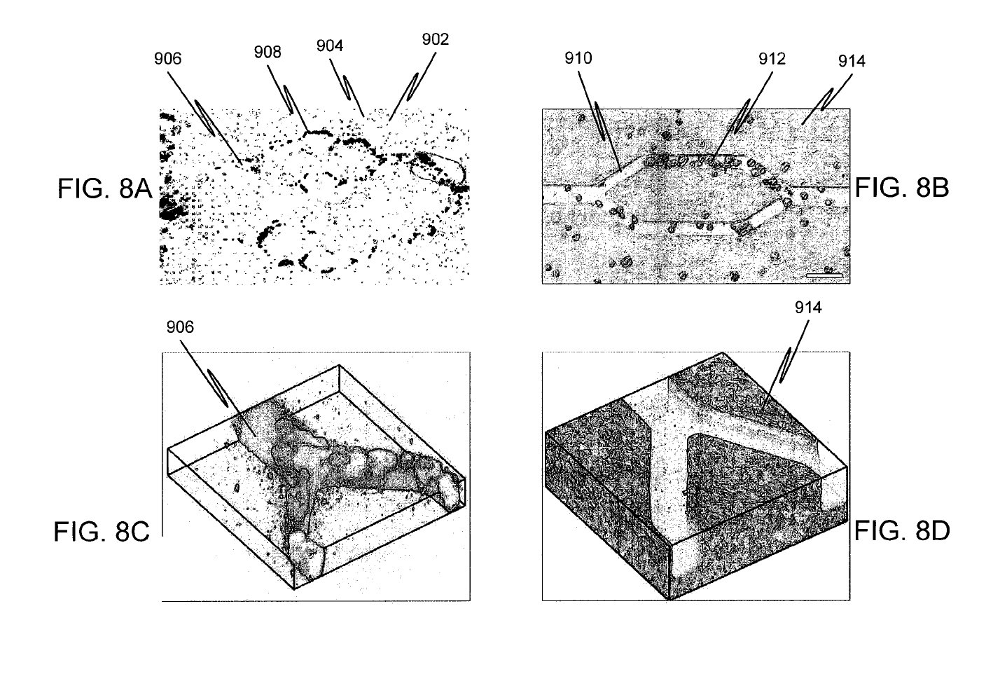

To investigate how collagen fiber assembly may integrate and anchor natural ECM phases against cell contraction, constructs consisting of pure alginate (FIGS. 8A and 8C) or collagen-doped alginate (FIGS. 8B and 8D) bulk phases (seeded with fibroblasts), and a collagen I matrix (seeded with human umbilical vein endothelial cells (HUVECs)) patterned within branched channel designs were fabricated. Fabrication of the composite collagen-alginate bulk phase employs a specific sequence of gelling. First, collagen is gelled by increasing temperature, followed by the gelling of alginate by ionic crosslinking. The pore size of crosslinked alginate permits the formation of a network of collagen fibers.

Immediately following fabrication, fibroblasts were uniformly distributed in the bulk phase and HUVECs were spatially restricted to the micropatterned phase (FIGS. 8A and 8B). The individual phases may be visualized by imaging collagen fibers in the constructs using confocal reflectance microscopy, followed by 3D reconstruction. Patterned HUVEC-seeded collagen was spatially restricted to and completely filled the microchannels inside the bulk alginate hydrogel (and was not merely coating the microchannel walls) (FIG. 8C).

In FIG. 8C, the 3D reconstruction of microfluidically patterned collagen seeded with HUVECs in a bare alginate bulk phase illustrates that HUVEC-seeded collagen completely filled the channels (as opposed to coating the walls) and that the phases were separated by the intended sharp boundaries. A subvolume of the entire stack is shown to better visualize the presence of HUVECs.

Moreover, visualization of open channels inside a collagen-doped alginate bulk phase confirmed a high fidelity in the replication of the mold, with collagen fibers uniformly distributed within the bulk phase (FIG. 8D). In FIG. 8D, the 3D reconstruction of the confocal z series through a collagen-alginate bulk phase before microfluidic patterning of collagen shows a qualitatively uniform distribution of collagen fibers and sharp channel boundaries with dimensions in agreement with the original master. Channel widths were 60 and 40 μm for both the master and the hydrogel scaffold, whereas the channel height was 56 μm for the master and 52 μm for the hydrogel scaffold. Separate phases may thus be patterned with at least a resolution of approximately 20 μm.

The collagen-alginate composite bulk phase and the micropatterned 3D collagen phase can also be used to regulate encapsulated cell morphology and behavior in composite hydrogel 3D constructs. For example, to permit spreading of fibroblasts encapsulated in the collagen-alginate bulk phase, the ionic crosslinks in alginate can be selectively (and reversibly) removed by applying sodium citrate, a calcium chelator, to the construct. The cells can then spread within the gelled porous collagen matrix.

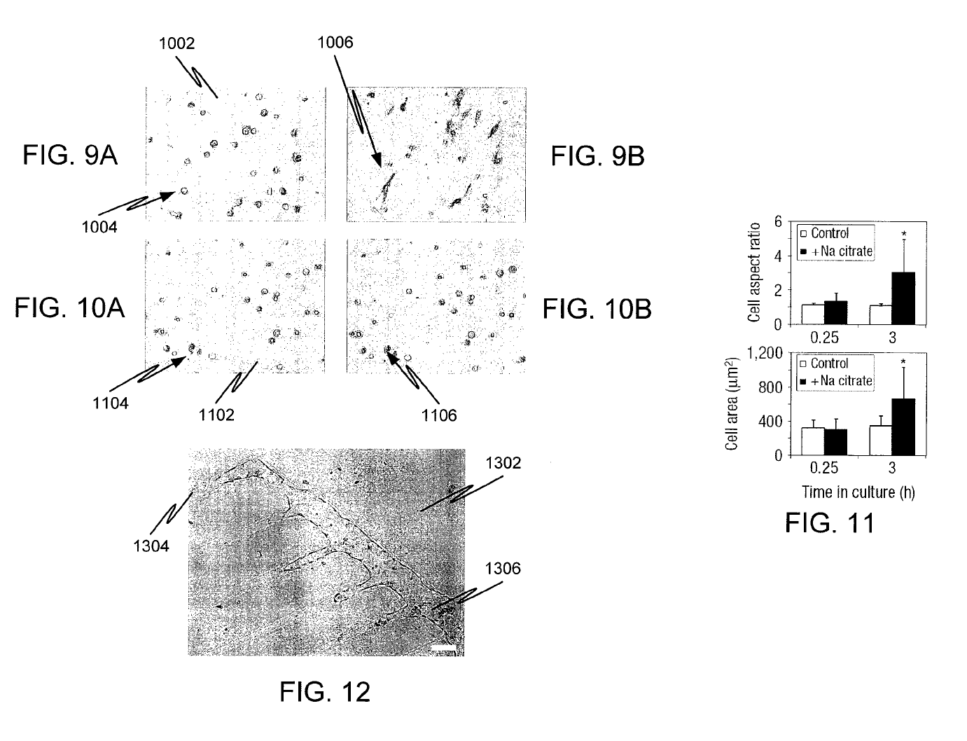

FIG. 9A is an image showing the morphology of fibroblasts 1004 inside a collagen-doped alginate phase 1002 at 15 minutes after exposure of the alginate phase to a calcium chelator. FIG. 9B is an image showing the morphology of fibroblasts 1006 inside a collagen-doped alginate phase 1002 at 3 hours after exposure of the alginate phase to a calcium chelator. After 3 hours, the fibroblasts 1006 had spread out and elongated in the uncrosslinked alginate-collagen matrix 1002, as determined by measurements of cell area and cell aspect ratio (FIG. 11).

FIG. 10A shows the morphology of fibroblasts 1104 inside a collagen-doped alginate phase 1102 at 15 minutes after culture without exposure to a calcium chelator. FIG. 10B shows the morphology of fibroblasts 1106 inside a collagen-doped alginate phase 1102 at 3 hours after culture without exposure to a calcium chelator. In contrast, the fibroblasts 1106 remained rounded within a collagen-alginate matrix 1102 that was not exposed to sodium citrate due to the nanometer-scale pore size of the crosslinked alginate. As reflected in the data of FIG. 11, 97% (±2%) of fibroblasts spread out after exposure to sodium citrate. No fibroblasts spread out in the unexposed constructs after 3 hours of culture.

This micropatterning capability can be used to restrict the formation of multicellular HUVEC structures inside 3D collagen of defined geometries, by using a dense, crosslinked alginate in the bulk phase, which also restricted fibroblast spreading in the bulk phase. FIG. 12 illustrates that HUVECs in the patterned phase 1306 did not enter the collagen-doped alginate bulk phase 1302 over 22 days of culture. Furthermore, large multicellular structures developed in a manner that conformed to the intended spatial geometry of the channels 1304. Dashed lines indicate channel boundaries 1304. Thus, use of this combination of bulk and patterned matrices permitted the spatial restriction in the formation of multicellular structures to within designed geometries in a hydrogel 3D construct.

Two ECM types with different porosities and cell adhesive properties can be integrated into a single composite ECM. Cell movement within the integrated composite ECM can be dynamically altered by altering the matrix properties to permit or restrict cell movement within the scaffold. Specifically, a two-component ECM can be constructed in which one component is fibrous and cell-adhesive (e.g., collagen I) and the other component is a non-adhesive polysaccharide that can be reversibly crosslinked by divalent cations (e.g., alginate). By controlling the state of ionic crosslinks in the non-adhesive polysaccharide component, the matrix porosity and adhesion properties of the composite ECM can be reversibly switched in order to restrict or permit cell spreading and migration.

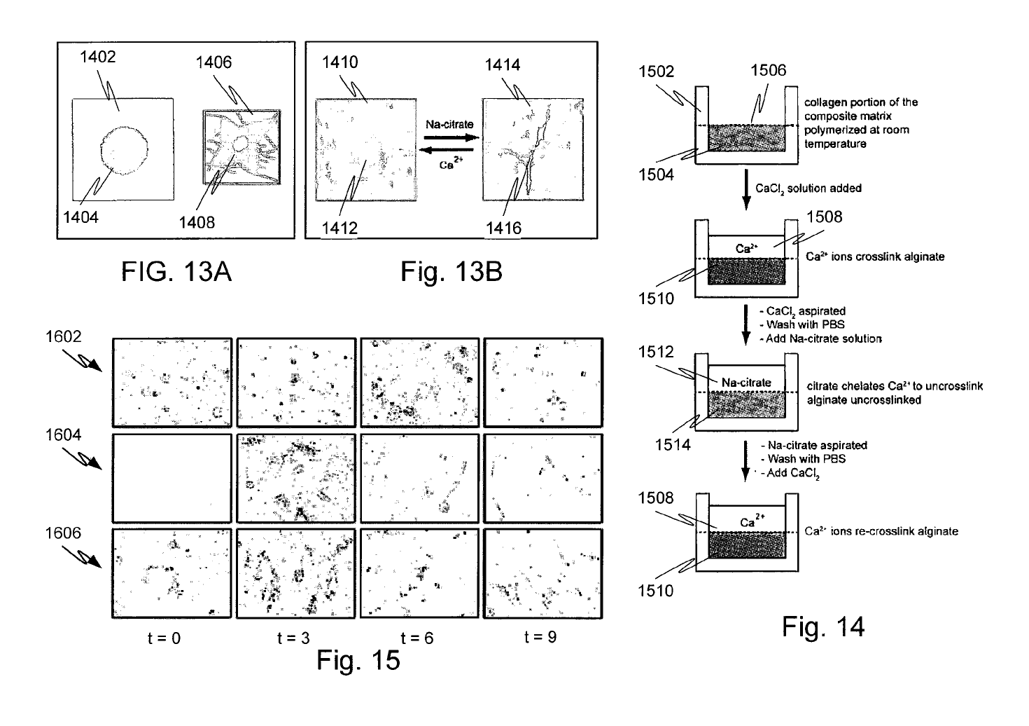

The ability of cells to spread and migrate within a 3D matrix depends largely on the matrix porosity and cell adhesive properties of the ECM material. As shown in FIG. 13A, collagen I is a naturally derived ECM that self-assembles into highly porous fibrous matrices 1406 that are also conducive to cell adhesion. Similarly, high porosity and cellular adhesion can be achieved with synthetic ECMs produced by techniques such as electrospinning. Cells 1408 in such matrices 1408 demonstrate a highly mobile behavior. Other naturally derived and synthetic ECM hydrogels 1402, such as agarose, alginate and polyethylene glycol (PEG), support viability of adherent cell types, but may not permit 3D spreading and migration, unless specifically engineered to do so. As a result, cells 1404 typically remain rounded and immobilized in such matrices 1402. Hydrogels of this type typically have much smaller pore sizes than fibrous ECM.

Compared to collagen, which polymerizes irreversibly to form a gel, alginate and agarose gel reversibly by means of ionic crosslinking or temperature crosslinking, respectively. As shown in FIG. 13B, it is possible to combine both classes of 3D matrix materials to permit cell spreading and migration in the microporous cell-adhesive ECM component (e.g., collagen I) while the reversibly gelled nanoporous non-adhesive ECM component (e.g., alginate) can be used to selectively immobilize cells. By combining functionalities of both matrix types, the composite ECM can support dynamic switching between cell immobilization state 1410 and spreading state 1414 to allow a cell 1412 to spread, migrate, and/or differentiate into cell 1416.

A method to dynamically modulate cell migration in a composite matrix is illustrated in FIG. 14. First, a cell-seeded collagen-alginate solution 1504 at cold temperature can be flowed into a well 1502. A membrane 1506 and a reservoir can be placed over the top surface. A fibrous collagen network can then be formed within the alginate solution by exposure to room temperature for 40 minutes.

The alginate can then be crosslinked and subsequently un-crosslinked by applying CaCl₂ solution and sodium citrate respectively. Crosslinking occurs when of Ca²⁺ 1508 diffuses across the membrane 1510 and binds with multiple alginate molecules. This creates a nanoporous matrix. The alginate component is crosslinked by introducing a calcium chloride solution across the membrane 1506 for 20 minutes, which is then aspirated off, washed with PBS, then replaced with culture medium.

Un-crosslinking can be achieved by the removal of these calcium ions by means of a chelator (i.e., citrate), thereby returning the matrix to a microporous state 1514. The alginate is uncrosslinked by introducing a sodium citrate solution 1512 across the membrane 1506 for 20 minutes. Aspiration, washing, and replacement with culture medium can follow.

The porosity of the matrix can therefore be controlled by alternatively applying the solutions as described. Immediately after treatment with either CaCl₂ or sodium citrate, the membranes can be washed with PBS and covered with Dulbecco's Modified Essential Medium (DMEM) for incubation.

To demonstrate dynamic temporal control of cell spreading and migration within the collagen-alginate composite matrix, the alginate component was uncrosslinked to permit spreading of cells, recrosslinked after 3 hours to halt spreading, then uncrosslinked again at 6 hours. The results are shown in image series 1606 of FIG. 15. In control groups in which the alginate was left crosslinked (image series 1602), cells remained rounded and immobilized, while cells continued to spread and migrate when the alginate was left uncrosslinked for the 6 hour duration (image series 1604).

In image series 1606 in FIG. 15, when the alginate component was re-crosslinked at 3 hours, cell spreading was halted until alginate was uncrosslinked again at 6 hours, at which point cells resumed spreading and migration. Cells in pure collagen gels continuously spread similar to uncrosslinked collagen-alginate gels, and cells in pure alginate gels remained immobilized similar to collagen-alginate gels that remain crosslinked. Thus, cell spreading and migration over multiple cycles can be selectively halted or permitted by dynamic control of ionic crosslinking of the alginate component of the composite matrix.

In addition to the temporal control of the matrix porosity, it is also possible to exert spatial control using high-resolution optical techniques. As shown in FIG. 19, photo-sensitive calcium caging molecules 1906—such as ethylene glycol tetraacetic acid (EGTA) and ethylenediaminetetraacetic acid (EDTA)—are incorporated into the matrix solution 1902 along with cells 1904. After crosslinking the matrix with a CaCl₂ solution, high resolution microscopy 1908 can selectively activate regions 1910 of the calcium cages 1906. These molecules 1906 chelate the Ca²⁺ ions from their bound state in the alginate 1902, thus creating small areas 1912 of microporous matrix within the bulk nanoporous gel 1902. Cells 1904 within this region 1912 can thus migrate and/or differentiate into cells 1914. The microporous region 1912 can be re-crosslinked by applying a CaCl₂ solution 1920 on top of a membrane 1918. This enables high spatial control of cell spreading and migration within the matrix.

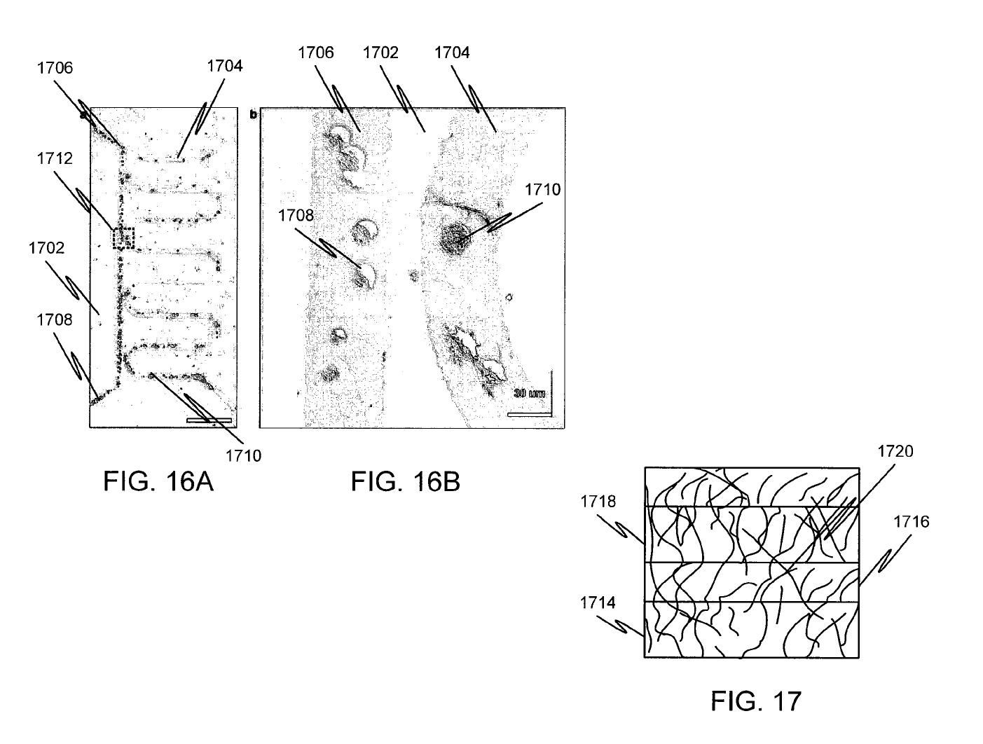

Multiple cell and ECM combinations can be rapidly flowed into multiple independent microchannels. Such a design is illustrated in FIG. 16A to pattern multiple ECMs within a single collagen-doped alginate bulk phase 1702. Fibroblasts 1708 were flowed into the straight channel 1706 while HUVECs 1710 were flowed into the curved channel 1704. A close-up of region 1712 is shown in FIG. 16B. The two cell types 1708, 1710 in their respective 3D collagen phases 1706, 1704 were patterned within 40 μm, with the alginate barrier 1702 between collagen phases being approximately 20 μm wide at the narrowest point.

FIG. 17 is a schematic diagram of a pattern for a construct of integrated matrix phases with multiple layers. The construct can be achieved by nucleating fibers in a second liquid 1716 from fibers in a gelled layer. The gelled layer 1714 may be previously formed by flowing a first liquid containing a gel precursor and a fibrilizing material and then gelling the liquid. This can be done in successive stages to form a construct having multiple layers, such as layer 1714, 1716, and 1718. Preferably, in such a configuration, some of the fibers 1720 can be built mechanically across multiple layers. The fibrilizing agent may be fibrin or collagen.

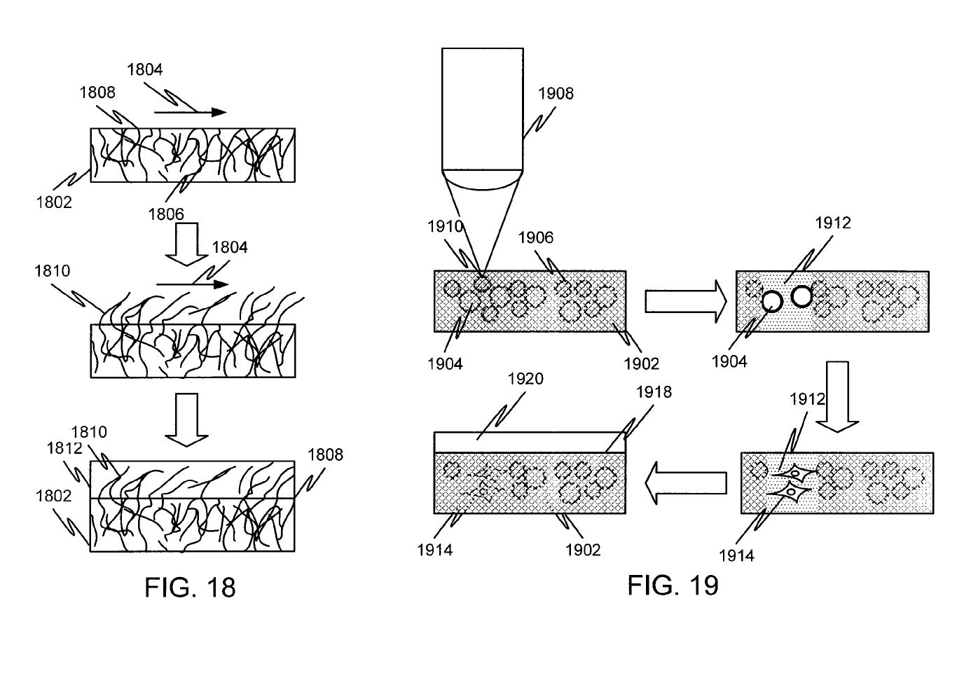

It may be desirable to have a gel with oriented fibers in it. Referring to FIG. 18, oriented fibers 1810 may be achieved by flowing a liquid containing a gel precursor and a fibrilizing agent in direction 1804 at the boundary 1808 of a material 1802. Material 1802 has preformed fibers 1806 contained therein. The preformed fibers 1806 at the boundary 1808 serves as a nucleation template for the growth of fibers in the adjacent matrix phase. Thus, fibers 1810 are grown from nucleating sites in the boundary 1808 into a space where the liquid flows. The rate of flow may be adjusted to achieve a desired orienting effect. For example, the flow may be slower than, or nearly as slow as, the rate of growth of the fibers grown from the fibrilizing material or faster than the rate of growth. When the gel precursor is gelled to form gel 1812, the fibers 1810 retain their orientation, as shown in FIG. 18.

A medical material including a gel precursor with a fiber material (e.g., collagen) may also be used as a tissue adhesive or wound dressing. The material may be provided in a sterile premixed form as a liquid and packaged with a sterile gelling agent or device. An agent capable of breaking down tissue can be used as a preparation to expose more nucleation sites. For example, collagenase can be used for this purpose. The gel precursor and fiber precursor may then be applied to the tissue after an effective period of time following the application of the agent. Then, after a period of time required for nucleation and fibrilization of the fiber material, the gel precursor may be gelled.

The foregoing descriptions apply, in some cases, to examples generated in a laboratory, but these examples can be extended to production techniques, which are preferred for implementing the invention. For example, where quantities and techniques apply to the laboratory examples, they should not be understood as limiting the claims.

It is, therefore, apparent that there is provided, in accordance with the present disclosure methods and systems for forming a biocompatible material. Many alternatives, modifications, and variations are enabled by the present disclosure. Features of the disclosed embodiments can be combined, rearranged, omitted, etc. within the scope of the invention to produce additional embodiments. Furthermore, certain features of the disclosed embodiments may sometimes be used to advantage without a corresponding use of other features. Accordingly, Applicant intends to embrace all such alternatives, modifications, equivalents, and variations that are within the spirit and scope of this invention

1. A method of forming a biocompatible material, comprising:
contacting a liquid, having a linking material, with an adjoining material having embedded therein a nucleating material that causes the linking material to nucleate and grow into the liquid; and,
after a time sufficient to cause the linking material to grow substantially from the nucleating material into a space occupied by the liquid, solidifying the liquid to form a solid such that the linking material secures the solid to the adjoining material.

2. The method of claim 1, wherein the solid is a gel and the solidifying includes gelling the liquid.

3. The method of claim 1, wherein the liquid and the adjoining material include at least one culture medium and live cells.

4. The method of claim 1, wherein the liquid, and the solid formed therefrom, include at least one of collagen, fibrin, alginate, and Matrigel®.

5. The method of claim 1, wherein the linking material includes at least one of engineered and natural fiber macromolecules.

6. The method of claim 1, wherein the linking material includes at least one of collagen, fibrin, elastin, laminin, and fibronectin.

7. The method of claim 1, wherein the nucleating material is the same essential molecular structure as the linking material.

8. The method of claim 1, wherein the linking and nucleating materials include collagen.

9. The method of claim 1, wherein the adjoining material includes living tissue.

10. The method of claim 1, wherein the adjoining material and the solid are essentially the same composition having different cell types.

11. The method of claim 1, further comprising contacting a further liquid, having a linking material, with the solid such that the linking material in the solid nucleates the linking material in the further liquid and solidifying the further liquid to form a further solid.

12. The method of claim 1, wherein the space occupied by the liquid includes a flow channel and the contacting includes flowing the liquid through the flow channel.

13. The method of claim 12, wherein the flow channel is part of a channel network and has a width of less than 100 microns.

14. A method forming a biocompatible material, comprising:
providing at least one gel body containing first cells and fibers forming a scaffold within the gel body, the fibers joining the gel body to an adjoining material; and
reversibly altering a property of a gel material forming the at least one gel body to permit the first cells to grow and/or move while the first cells continue to be supported by the fibers forming the scaffold.

15. The method of claim 14, further comprising restoring the property of the gel material forming the at least one gel body after an interval required for the growth and/or movement of the first cells.

16. The method of claim 14, wherein the property includes a crosslinking of material forming the gel material of the at least one gel body.

17. The method of claim 14, wherein the gel material includes one of alginate and agarose.

18. The method of claim 14, wherein the gel material includes alginate and the reversibly altering includes applying calcium chloride to the at least one gel body and the restoring includes applying a calcium chelator to the at least one gel body.

19. The method of claim 14, wherein the at least one gel body is one of multiple layers of gel.

20. The method of claim 14, wherein the adjoining material is a gel of a different material from the at least one gel body.

21. The method of claim 14, wherein the adjoining material is a gel containing second cells of a different type from the first cells.

22. The method of claim 14, wherein the property includes a mechanical property.

23. A biocompatible material, comprising:
a first gel material having at least one channel therein; and
a second gel material in the at least one channel,
wherein the second gel material is joined to the first gel material at an interface therebetween by fibers.

24. The biocompatible material of claim 23, wherein at least the second gel material contains living cells.

25. The biocompatible material of claim 23, wherein the fibers extend through the interface into the first and second gel materials to join the first and second gel materials together.

26. The biocompatible material of claim 23, wherein a substantial fraction of fibers extending through the interface in the first and second gel materials are directed at angles of approximately 90 degrees relative to the interface.

27. The biocompatible material of claim 23, wherein at least a portion of the at least one channel has a width less than 100 microns.

28. The biocompatible material of claim 23, wherein the at least one channel includes a network of channels.

29. The biocompatible material of claim 23, wherein the first and second gel materials are of different materials.

30. The biocompatible material of claim 23, wherein the first and second gel materials have different respective live cells.

31. A method for linking a matrix phase to an adjoining material, comprising:
flowing a liquid gel precursor containing a fibrilizing material adjacent the adjoining material, the adjoining material having, at an interface between the liquid and the adjoining material, a nucleating material which is adapted to nucleate growth of the fibrilizing material;
assembling fibers in the liquid gel precursor at the interface to form a fiber network in the liquid gel precursor; and
thereafter gelling the liquid gel precursor.

32. The method of claim 31, wherein the gel has living cells therein.

33. The method of claim 31, wherein the adjoining material includes living tissue.

34. The method of claim 31, wherein the gel precursor includes a hormone effective to stimulate a change in growth of living cells in the adjoining material.

35. The method of claim 31, wherein the adjoining material includes living tissue, the method further comprising, enzymatically treating the living tissue to enhance the exposure of the nucleating material to the fibrilizing material.

36. The method of claim 31, wherein the adjoining material includes living tissue, the method further comprising, prior to the flowing, enzymatically treating the living tissue to enhance the exposure of the nucleating material to the fibrilizing material.

37. The method of claim 31, wherein the adjoining material is a gel including a network of microchannels and the flowing includes flowing the liquid gel precursor through at least a portion of the network.

38. The method of claim 31, wherein the nucleating material in includes existing fibers, of the same material as the fibrilizing material, extending into the adjoining material, the assembling including extending the existing fibers to form the fiber network.

39. A medical treatment kit, comprising:
materials that may be combined to form, or, a liquid composition containing a gel precursor and soluble collagen; and
a biocompatible gelling agent effective to solidify the gel precursor.

40. A kit as in claim 39, further comprising an enzyme effective to enhance the exposure of collagen in living tissue to act as nucleation sites for the soluble collagen.

41. A biocompatible material, comprising:
a first material of a first composition;
a second material of a second composition adjacent to and in contact with the first material; and
fibers extending between the first and second materials across a first interface therebetween, wherein the first material is principally a hydrogel.

42. The biocompatible material of claim 41, further comprising a third material in contact with the first material on a side opposite the first interface in which the fibers continue across a second interface between the first and third materials.

43. The biocompatible material of claim 41, wherein the second material includes living tissue.

44. The biocompatible material of claim 41, wherein the first material includes living cells, the fibers having a number, type, and orientation effective to prevent detachment of the first interface in response to a growth of the living cells.

45. The biocompatible material of claim 41, further comprising a third material adjacent to and in contact with the second material at a second interface therewith and fibers extending between the second and third materials across the second interface.

46. The biocompatible material of claim 41, wherein a substantial fraction of the fibers extending across the first interface have angles of approximately 90 degrees with respect to the first interface.

47. A method of forming a biocompatible material, comprising:
stemming fibers in at least one liquid containing a soluble fibrilizing material from fibers in an adjacent material at least one surface adjacent to the at least one liquid such that the fibers are continuous across the at least one surface from the adjacent material to the at least one liquid; and
solidifying the at least one liquid to hold fast, stemmed fibers in solidified liquid resulting from the solidifying.
